(19) United States
(12) Patent Application Publication
(10) Pub. No.: US 2019/0151394 A1
(43) Pub. Date: May 23, 2019

(54) Prebiotic Acne Treatment

(71) Applicant: Ying Tung Chen, Sunnyvale, CA (US)

(72) Inventor: Ying Tung Chen, Sunnyvale, CA (US)

(21) Appl. No.: 15/816,742

(22) Filed: Nov. 17, 2017

(51) Int. Cl. A61K 36/82; A61K 9/00; A61P 17/10; A61K 36/484; A61K 36/28; A61K 36/23; A61K 36/70; A61K 36/539; A61K 36/53; A61K 36/185; A61K 31/765; A61K 31/047; A61K 31/4166; A61K 31/194; A61K 31/192

(52) U.S. Cl. CPC A61K 36/82; A61K 31/192; A61P 17/10; A61K 36/484; A61K 36/28; A61K 36/23; A61K 36/70; A61K 36/539; A61K 36/53; A61K 36/185; A61K 31/765; A61K 31/047; A61K 31/4166; A61K 31/194; A61K 9/0014

(57) ABSTRACT

A topical composition comprising an effective amount of a prebiotic capable of nourishing and encouraging skin microorganisms such as S. epidermidis to colonize the skin and supplant acne-causing bacteria such as P. acnes. Additional components can be used by S. epidermidis to create short chain fatty acids that inhibit growth of P. acnes. The preferred prebiotics are Camellia Sinensis Leaf Extract, Glycyrrhiza Glabra (Licorice) Root Extract, and Chamomilla Recutita (Matricaria) Flower Extract. PEG-12 is used by S. epidermidis to create short chain fatty acids that inhibit P. acnes. The composition is highly effective in a method of treating acne.

### FIELD OF THE INVENTION

The present invention relates to topical compositions and methods for treatment of acne.

### BACKGROUND OF THE INVENTION

Acne is an inflammatory skin condition arising from disorders of pilosebaceous units, which includes a hair follicle, sebaceous gland, and a hair.

Four primary factors that lead to the formation of acne vulgaris, and include: (1) increased sebum output resulting in oily, greasy skin; (2) increased bacterial activity normally due to an overabundance of Propionibacterium acnes bacteria; (3) plugging (hypercornification) of the follicle or pilosebaceous duct; and (4) inflammation triggered by substances leaking into the dermis which cause inflammatory reactions. The plugged pores result in blackheads, whiteheads, pimples or deeper lumps such as cysts or nodules.

It is generally believed that the presence of high levels of Propionibacterium acnes (P. acnes) in the pilosebaceous unit is one of the causes of the inflammation associated with acne. P. acnes produce a lipase enzyme that hydrolyzes triglycerides of the sebaceous gland into free fatty acids. The fatty acids along with bacterial proteins and keratin irritate the skin tissues. This may lead to an inflammatory response and the formation of an acne lesion. The presence of P. acnes attracts white blood cells which act on the pilosebaceous unit, which manifests as the papules (red bumps), pustules, and nodules typical of acne. Acne lesions may further be triggered by a combination of P. acnes and coagulase-negative staphylococci and micrococci.

Various therapeutic methods for treating acne have been proposed. A very conventional approach is the use of topical antibacterials. Benzoyl peroxide (BP), typically at 5%, has antibacterial activity and drying effects and is available over the counter or by prescription. BP is applied once or twice daily but may take 1-2 months to have an effect. BP is often tried first for both non-inflammatory and mild inflammatory acne. Salicylic acid has a similar efficacy.

Topical use of hexachlorophene was popular in the 1960s and 1970s but its use has been discontinued as the treatment was neither highly effective and it also posed the risk of promoting antibiotic-resistant microbes and thereby causing significant public health risks.

Combinations such as BP (5%) and erythromycin (3%) are available by prescription. Other topical antibiotics include clindamycin and erythromycin are also available by prescription.

Vitamin A, in the form of topical retinoic acid or tretinoin, both derivatives of vitamin A, have been used to treat acne topically. These topical agents work by normalizing the skin's production of keratin and the sebaceous glands production of sebum, thereby preventing obstruction of the follicle. Although effective, Vitamin A topical treatments often take several months to have an effect. The patient's condition may become worse before it improves. Side effects include stinging and reddening of the treated areas and possible photosensitivity.

Systemic treatments for acne include the use of oral antibiotics. These treatments are directed towards the reduction in the amount P. Acnes in the skin, especially the pilosebaceous structures, and seek to reduce the inflammation caused by waste materials and metabolic byproducts from these organisms. Tetracycline antibiotics are most commonly used for this purpose. These include tetracycline, minocycline and doxycycline. Erythromycinis also sometimes used. In cases where acne does not respond to oral antibiotic treatment, oral isotretinoin is sometimes used. While effective, isotretinoin is also powerfully teratogenic, and women of childbearing age are required to use multiple methods of contraception while taking the drug.

Accordingly, an effective treatment of acne, particularly its more severe forms, with clinically insignificant side effects is desired.

### SUMMARY OF THE INVENTION

The present invention relates to a topical composition comprising an effective amount of a selected prebiotic capable of nourishing and encouraging skin microorganisms that will colonize the skin and supplant acne-causing bacteria such as P. acnes, and additional components that such skin microorganisms can use to suppress acne-causing bacteria such as P. acnes, in a dermatologically acceptable carrier.

The preferred prebiotics are Camellia Sinensis Leaf Extract, Glycyrrhiza Glabra (Licorice) Root Extract, and Chamomilla Recutita (Matricaria) Flower Extract which facilitate a healthy Staphylococcus epidermidis (S. epidermidis) population on the skin. Additionally, PEG-12 in the composition delivers the molecular raw material used by S. epidermidis to create short chain fatty acids that inhibit P. acnes. Thus, the topical composition provides S. epidermidis with raw materials used support the S. epidermidis and to produce short chain fatty acids that suppress P. acnes.

The present invention also relates to a method of treating, improving the appearance of, and/or preventing acne including, but not limited to mild, moderate, severe, comedonal, inflammatory, pustular, or cystic acne in a human comprising administering to the human a composition comprising an effective amount of a selected prebiotic capable of nourishing and encouraging skin microorganisms that will colonize the skin and supplant or suppress acne-causing bacteria such as P. acnes, in a dermatologically acceptable carrier.

Topical compositions according to the present invention may be formulated, for example, in a cream, ointment, lotion, gel, foam, solution, or cleanser form.

### DETAILED DESCRIPTION OF THE INVENTION

The present invention comprises a composition and a method for prevention and/or treatment of acne that is based on the principle of providing probiotic agents that will nourish and encourage skin microorganisms that will colonize the skin and supplant acne-causing bacteria such as P. acnes, combined with providing agents that will aid in actively suppressing P. acnes.

The presence or absence of particular skin microorganisms is dependent on numerous factors including temperature; humidity; ultraviolet exposure; diet; clothing choice; use of antibiotics, antibacterial soaps, moisturizers, cosmetics, hand sanitizers, and/or other anti-microbial skin products. Some skin microorganisms provide benefits to their human host, for example, by stimulating the human immune system and/or producing anti-microbial substances. Beneficial microorganisms that have been recognized are saprophyte microorganisms and/or coagulase-negative staphylococci, in particular, S. epidermidis, S. hominis, S. warneri, S. saprophyticus, S. xylosus, S. capitis or S. simulans. Of these, S. epidermidis and S. warneri are considered most beneficial. For example, S. epidermidis has been stated to produce anti-microbial peptides that inhibit Staphylococcus aureus biofilm formation.

The present invention is directed at the use of agents that provide prebiotic activity for those members of the skin microbiome that produce a benefit to the host, and further at components that can be used by the skin microbiome to suppress acne-causing bacteria. In particular, the topical composition encourages the proliferation of S. epidermidis.

Non-beneficial microorganisms found in the skin include coagulase positive Staphylococci, in particular S. aureus, P. acnes, Corynebacterium spp., Peptostreptococcus spp., Candida albicans, and Malassezia furfur. P. acnes in particular is associated with facial acne.

The present invention comprises a composition and a method for prevention and/or treatment of acne that is based on the principle of providing probiotic agents that will nourish and encourage skin microorganisms that will colonize the skin and supplant acne-causing bacteria such as P. acnes, combined with providing agents that will aid beneficial bacteria in actively suppressing P. acnes, by encouraging the production by S. epidermidis of short chain fatty acids that suppress P. acnes.

Compositions containing such agents are prepared by combining the agents with an acceptable dermatological carrier and used topically. For example, moisturizers, hand and/or body soaps, cosmetics, hand sanitizers, body lotions, and/or other skin products suitable for human use may be formulated to include such agents.

A preferred embodiment of a composition in accordance with the invention comprises Water (Aqua), Butylene Glycol, PEG-12, Allantoin, Propylene Glycol, Cucumis Sativus Fruit Extract, Dipotassium Glycyrrhizate, Centella Asiatica Extract, Polygonum Cuspidatum Root Extract, Scutellaria Baicalensis Root Extract, Camellia Sinensis Leaf Extract, Glycyrrhiza Glabra (Licorice) Root Extract, Chamomilla Recutita (Matricaria) Flower Extract, Rosmarinus Officinalis (Rosemary) Leaf Extract, Buddleja Davidii Leaf Extract, Glycerin, Citric Acid, Sodium Benzoate, and Potassium Sorbate.

In the above described preferred embodiment, the prebiotic agents are Camellia Sinensis Leaf Extract, Glycyrrhiza Glabra (Licorice) Root Extract, and Chamomilla Recutita (Matricaria) Flower Extract. It is believed that these ingredients work in conjunction with each other to support the S. epidermidis on the skin. The embodiment further includes PEG-12 which is the molecular raw material used by S. epidermidis to create short chain fatty acids that inhibit P. acnes.

A specific preferred embodiment of a composition in accordance with the invention comprises the following:

| Composition Ingredients | Weight % of composition |
|---|---|
| Water(Aqua) | 40%-60% |
| Butylene Glycol | 20%-35% |
| PEG-12 | 0.5%-3.0% |
| Allantoin | 0.1%-1.5% |
| Water(Aqua) | The phase/combination |
| Propylene Glycol | is 0.5%-2.5% |
| Cucumis Sativus Fruit | |
| Extract | |
| Dipotassium Glycyrrhizate | 0.05%-.5% |
| Butylene Glycol | The phase/combination |
| Water(Aqua) | is 5%-25% |
| Centella Asiatica Extract | |
| Polygonum Cuspidatum | |
| Root Extract | |
| Scutellaria Baicalensis Root | |
| Extract | |
| Camellia Sinensis Leaf | |
| Extract | |
| Glycyrrhiza Glabra | |
| (Licorice) Root Extract | |
| Chamomilla Recutita | |
| (Matricaria) Flower Extract | |
| Rosmarinus Officinalis | |
| (Rosemary) Leaf Extract | |
| Buddleja Davidii Extract | The phase/combination |
| Glycerin | is 0.5%-5% |
| Water(Aqua) | |
| Citric Acid | |
| Sodium Benzoate | |
| Potassium Sorbate | |
| Total | 100 |

In preferred embodiments, the Camellia Sinensis Leaf Extract is 0.05%-0.5 weight % of the composition, most preferably 0.05%-0.25% by weight of the composition.

In preferred embodiments, the Glycyrrhiza Glabra (Licorice) Root Extract is 0.05%-0.5 weight % of the composition, most preferably 0.05%-0.25% by weight of the composition.

In preferred embodiments, the Chamomilla Recutita (Matricaria) Flower Extract Root Extract is 0.05%-0.5 weight % of the composition, most preferably 0.05%-0.25% by weight of the composition.

In preferred embodiments, the PEG-12 is 0.05%-0.5 weight % of the composition, most preferably 0.5%-2.5% by weight of the composition.

The composition may optionally include sulfur. If included, sulfur may comprise 1%-10% w/w Sulfur USP or 5%-15% w/w sodium sulfacetamide in the composition. Another option is cruciferous sulforaphane, the sulforaphane C₆H₁₁NOS₂ compound found and/or extracted from a broad range of vegetables of the Brassica oleracea genus. Cruciferous sulforaphane may be present at about 0.1% to about 5.0% by weight, preferably, 0.5 to 2% by weight of a topical compositions.

Some embodiments of this invention contain at least one other adjunct active ingredients. Adjunct ingredients may be present in an amount ranging from 0.01% to about 20% by weight of the composition. They include, but are not limited to one or more of: caffeine, vitamin D3, lipoic acid; α-hydroxy acids such as glycolic acid or lactic acid; ascorbic acid and its derivatives, especially fatty acid esters of ascorbic acid; polyenylphosphatidylcholine; or tocotrienols and tocotrienol derivatives and vitamin E compositions enriched with tocotrienols or tocotrienol derivatives; and neuropeptides. Preferred adjunct agents include caffeine, tocopherol, tocopherol acetate, acetyl tyrosine, palmitoyl tripeptide-5, thiotic acid, hexylene glycol, magnesium ascorbyl phosphate and tetrahexyldecyl ascorbate.

The topical composition of the invention can contain ingredients other than the above that are commonly found in skin care compositions and cosmetics, such as, for example, tinting agents, emollients, skin conditioning agents, emulsifying agents, humectants, preservatives, antioxidants, perfumes, chelating agents, etc., provided that they are physically and chemically compatible with other components of the composition.

Preservatives are typically present in an amount ranging from about 0.1% to about 2.0% by weight percent, based on the total composition. Preferred preservatives include phenoxyethanol and/or sodium benzoate and/or potassium sorbate.

Emollients, typically present in amounts ranging from about 0.01% to 10% of the total composition include, but are not limited to, fatty esters, fatty alcohols, mineral oils, polyether siloxane copolymers, docosahexanoic acid (DHA) and mixtures thereof. Emollients may include isohexadecane, cetyl alcohol, cetearyl alcohol, stearyl alcohol, ethylhexyl palmitate, and avocado oil.

Humectants, typically present in amounts ranging from about 0.1% to about 5% by weight of the total composition include, but are not limited to, polyhydric alcohols such as glycerol, polyalkylene glycols (e.g., butylene glycol, propylene glycol, dipropylene glycol, polypropylene glycol, and polyethylene glycol) and derivatives thereof, alkylene polyols and their derivatives, sorbitol, hydroxy sorbitol, hexylene glycol, 1,3-dibutylene glycol, 1,2,6-hexanetriol, ethoxylated glycerol, propoxylated glycerol, and mixtures thereof. Preferred humectants are glycerin and glycol derivatives, such as hexylene glycol, ethylhexylglycerin, glyceryl stearate, peg-12 glyceryl dimyristate, and caprylyl glycol and shae butter.

Emulsifiers, typically present in amounts from about 1% to about 15% by weight of the composition, include, but are not limited to, polysorbagte 20, stearic acid, cetyl alcohol, stearyl alcohol, steareth 2, steareth 20, acrylates/C10-30 alkyl acrylate crosspolymers, silicones, dimethylethanolamine (DMAE), phosphatidylcholine (PPC), docosahexanoic acid (DHA) and mixtures thereof.

Chelating agents, typically present in amounts ranging from about 0.01% to about 2% by weight, include, but are not limited to, ethylenediamine tetraacetic acid (EDTA) and derivatives and salts thereof, dihydroxyethyl glycine, tartaric acid, and mixtures thereof.

Antioxidants, typically present in an amount ranging from about 0.01% to about 0.75% by weight of the composition, include, but are not limited to, butylated hydroxy toluene (BHT); vitamin C and/or vitamin C derivatives, such as fatty acid esters of ascorbic acid, particularly ascorbyl palmitate; butylated hydroanisole (BHA); phenyl-α-naphthylamine; hydroquinone; propyl gallate; nordihydroquiaretic acid; vitamin E and/or derivatives of vitamin E, including tocotrienol and/or tocotrienol derivatives; calcium pantothenates; and mixtures of any of these. Particularly preferred antioxidants are those that provide additional benefits to the skin, such as ascorbyl palmitate, alpha-lipoic acid, magnesium ascorbyl phosphate and tetrahexyldecyl ascorbate.

Optional ingredients include perfumes and fragrances such as citrus aurantium dulcis (orange) fruit extract, citrus limon (lemon) fruit extract and the like.

Buffering agents are not generally needed in the composition but may be used. Typical buffering agents are chemically and physically stable agents commonly found in cosmetics, and can include compounds that are also adjunct ingredients such as citric acid, malic acid, and glycolic acid buffers.

Generally in the practice of methods of the invention, the topical composition is topically applied to the skin areas, such as that of the face, at predetermined intervals often as a moisturizer, lotion, or cream, it generally being the case that improvement is noted with each successive application. Although immediate effects can be observed, enhanced results are observed when the topical composition is applied twice daily, preferably in the morning and evening. Insofar as has been determined based upon clinical studies to date, no adverse side effects are encountered. It is an advantage of the invention that compositions of the invention do not require a pharmaceutical prescription.

A clinical study was conducted on a topical acne treatment formulated according to the above embodiment. The acne treatment was prepared and tested on a panel of test subjects. Control panels of test subjects using a topical salicylic acid acne treatment, and a topical placebo were also tested.

The following test protocol was followed in the clinical study.

### Visit 1 (Baseline)

Subjects reported to the testing facility for screening. Informed Consent was obtained, demographics collected and study eligibility confirmed. Upon verification of general eligibility, subjects then had a visual evaluation of their face by a trained evaluator to confirm eligibility. The visual evaluations included assessments using the Global Visual Scale. Upon verification of eligibility, a subset of subjects (15 subjects from cell 1) were selected to have baseline photographs taken of their face using a professional photographer. All subjects were provided with test article and usage instructions for home use between the three visits.

### Visit 2 (Day 10)

Subjects returned to the test facility following 10 days of use, where any adverse events were reviewed and recorded. A visual evaluation by a trained evaluator of the face was conducted using the Global Visual Scale. Professional Photographs were taken of the selected 15 subjects.

### Visit 3 (Day 56)

Subjects returned to the test facility following 56 days of use for their final assessments. Any adverse events were reviewed and recorded. A visual evaluation by a trained evaluator of the face was conducted using the Global Visual Scale. Professional Photographs were taken of the selected 15 subjects. An online subjective Self Perception Questionnaire (SPQ) about the subject's experience using the test articles was completed and reviewed by study staff for completeness. At this time any unused products were returned.

Test subjects were instructed to apply the product (Product 2) twice each day—in the morning and before going to bed. In the case of the composition described in the application, test subjects were advised that a couple of drops of acne treatment was the appropriate dosage. The instructions also included instructions regarding use of a skin cleanser (Product 1) and a skin moisturizing cream (Product 3). The instructions stated:
1. Remove all facial products (makeup, sunscreen, etc.) with makeup remover.
2. Wet your face with warm water, squeeze bean-size amount of product 1 to your finger tips and massage in circular motion for 30 seconds.
3. Rinse off with warm water at least ten times. Make sure to wash away all of the product.
4. Apply product 2 on the affected area gently.
5. Once product 2 dries, apply and massage product 3 to the entire face.
6. Repeat 1-5 in the morning and before going to bed.

Visual evaluations of test subject acne were performed at baseline, day 10, and day 56 by a trained grader using the following Global Visual Scale:

### IGA Scale Description

0 Clear skin with no inflammatory or noninflammatory lesions
1 Almost clear; rare noninflammatory lesions with no more than one small inflammatory lesion
2 Mild severity; greater than Grade 1; some noninflammatory lesions with no more than a few inflammatory lesions (papules/pustules only, no nodular lesions)
3 Moderate severity; greater than Grade 2; up to many noninflammatory lesions and may have some inflammatory lesions, but no more than one small nodular lesion
4* Severe; greater than Grade 3; up to many noninflammatory and inflammatory lesions, but no more than a few nodular lesions

The 30 test subjects who used a composition in accordance with the present invention started with a mean Baseline Scale of 2.59, which after 10 days dropped to 1.3 (a 60% reduction) and after 56 days dropped to 0.31 (an 88% reduction from the baseline).

In comparison, the 30 test subjects who used a placebo lacking any anti-acne component started with a mean Baseline Scale of 2.6, which after 10 days increased to 2.63 (a 1.28% increase) and after 56 days increased to 2.67 (a 2.56% increase from the baseline).

The 30 test subjects who received a commercially available salicylic acid treatment started with a mean Baseline Scale of 2.7, which after 10 days dropped to 1.6 (a 40.74% reduction) and after 56 days dropped to 0.6 (an 77.78% reduction from the baseline).

Therefore, clinical data establishes that the present invention has a significant efficacy in resolving the appearance of acne. In the described study, the claimed invention performed better than an FDA-approved therapy, salicylic acid. Thus the present invention is efficacious, superior to conventional acne products on the market, and at the same time avoids harsher peroxides and acids currently used in acne medications.

What is claimed is:

1. A topical composition comprising an effective amount of a prebiotic capable of nourishing skin microorganisms to supplant acne-causing bacteria in skin.

2. The topical composition of claim 1, wherein the prebiotic is capable of nourishing skin microorganisms to supplant Propionibacterium acnes in skin.

3. The topical composition of claim 2, wherein the prebiotic is capable of nourishing Staphylococcus epidermidis to supplant Propionibacterium acnes in skin.

4. The topical composition of claim 3, wherein the prebiotic comprises Camellia Sinensis Leaf Extract, Glycyrrhiza Glabra (Licorice) Root Extract, and Chamomilla Recutita (Matricaria) Flower Extract.

5. The topical composition of claim 3, wherein the prebiotic comprises one or more of Camellia Sinensis Leaf Extract, Glycyrrhiza Glabra (Licorice) Root Extract, or Chamomilla Recutita (Matricaria) Flower Extract.

6. The topical composition of claim 4, wherein the composition further comprises Centella Asiatica Extract, Polygonum Cuspidatum Root Extract, Scutellaria Baicalensis Root Extract, Camellia Sinensis Leaf Extract, Glycyrrhiza Glabra (Licorice) Root Extract, Chamomilla Recutita (Matricaria) Flower Extract, Rosmarinus Officinalis (Rosemary) Leaf Extract, and Buddleja Davidii Leaf Extract.

7. The topical composition of claim 5, wherein the composition further comprises one or more of Centella Asiatica Extract, Polygonum Cuspidatum Root Extract, Scutellaria Baicalensis Root Extract, Camellia Sinensis Leaf Extract, Glycyrrhiza Glabra (Licorice) Root Extract, Chamomilla Recutita (Matricaria) Flower Extract, Rosmarinus Officinalis (Rosemary) Leaf Extract, or Buddleja Davidii Leaf Extract.

8. The topical composition of claim 3, wherein the composition further comprises molecular components that Staphylococcus epidermidis can use to create short chain fatty acids that inhibit Propionibacterium acnes.

9. The topical composition of claim 8, wherein the molecular components that Staphylococcus epidermidis can use to create short chain fatty acids is PEG-12.

10. The topical composition capable of nourishing Staphylococcus epidermidis to inhibit Propionibacterium acnes in skin, comprising:
Water (Aqua);
PEG-12;
Cucumis Sativus Fruit Extract;
Dipotassium Glycyrrhizate;
Centella Asiatica Extract;
Polygonum Cuspidatum Root Extract;
Scutellaria Baicalensis Root Extract′
Camellia Sinensis Leaf Extract;
Glycyrrhiza Glabra (Licorice) Root Extract;
Chamomilla Recutita (Matricaria) Flower Extract;
Rosmarinus Officinalis (Rosemary) Leaf Extract; and
Buddleja Davidii Leaf Extract.

11. The topical composition of claim 10, further comprising: Butylene Glycol; Propylene Glycol; and Allantoin.

12. The topical composition of claim 11, further comprising: Glycerin, Citric Acid, Sodium Benzoate, and Potassium Sorbate.

13. A method of preventing or treating acne in a human comprising:
topically administering to the human a composition comprising an effective amount of a prebiotic capable of nourishing and encouraging skin microorganisms that will colonize the skin and supplant Propionibacterium acnes in a dermatologically acceptable carrier.

14. The method of claim 13, wherein the prebiotic comprises Camellia Sinensis Leaf Extract, Glycyrrhiza Glabra (Licorice) Root Extract, and Chamomilla Recutita (Matricaria) Flower Extract.

15. The method of claim 14, wherein the composition further comprises Centella Asiatica Extract, Polygonum Cuspidatum Root Extract, Scutellaria Baicalensis Root Extract, Camellia Sinensis Leaf Extract, Glycyrrhiza Glabra (Licorice) Root Extract, Chamomilla Recutita (Matricaria) Flower Extract, Rosmarinus Officinalis (Rosemary) Leaf Extract, and Buddleja Davidii Leaf Extract.

16. The method of claim 14, wherein the composition further comprises PEG-12.

17. The method of claim 14, wherein a skin microorganism that will colonize the skin and supplant Propionibacterium acnes is Staphylococcus epidermidis.

18. The method of claim 16, wherein the Staphylococcus epidermidis converts the PEG-12 to short chain fatty acids which inhibit Propionibacterium acnes.
